(19) United States
(12) Patent Application Publication
(10) Pub. No.: US 2008/0167684 A1
(43) Pub. Date: Jul. 10, 2008

(54) Treatment table with calf/foot assembly and method of use

(76) Inventor: David F. Cuccia, Syosset, NY (US)

Correspondence Address: MELVIN K. SILVERMAN AND ASSOCS PC, 500 WEST CYPRESS CREEK ROAD, SUITE 350, FT. LAUDERDALE, FL, 33309, US

(21) Appl. No.: 12/002,446

(22) Filed: Dec. 17, 2007

Related U.S. Application Data
(63) Continuation-in-part of application No. 11/189,956, filed on Jul. 25, 2005, now Pat. No. 7,309,347.
Application No. 11/189,956 is a continuation of application No. 10/413,730, filed on Apr. 15, 2003, now Pat. No. 6,923,825.

(51) Int. Cl. A61F 5/00

(52) U.S. Cl. 606/242; 606/241; 602/32

(57) ABSTRACT

A treatment table with a calf/foot assembly includes an upper body support section adapted to support a patient's body, attached to a system support; and a calf/foot assembly pivotally attached to a lower part of the upper body support section, the calf/foot assembly including a calf/foot support, a linear movement mechanism adapted to move the calf/foot support linearly, and a first rotational mechanism adapted to cause a first transverse movement of the calf/foot support transverse to the longitudinal axis of the upper body support section. The treatment table further includes a second rotational mechanism adapted to cause a second transverse movement of the calf/foot support transverse to the longitudinal axis and the first transverse movement. The method of using the treatment table to applying traction to the spine of a patient at varying angles is provided.

### CROSS REFERENCE TO RELATED APPLICATION

This application is continuation-in-part of application Ser. No. 11/189,956, filed on Jul. 25, 2005 which is a continuation of patent application Ser. No. 10/413,730, filed Apr. 15, 2003, now U.S. Pat. No. 6,923,825. All prior applications are hereby incorporated by reference in their entirety.

### BACKGROUND OF THE INVENTION

Back pain is a common condition. One commonly used non-surgical approach to alleviating back pain in patients is the application of traction forces. Traction tables are used to apply traction forces to the human body through the application of tension force along the spinal column.

It is to be appreciated that the success of any device designated to treat lower back dysfunction is in large part dependent on proper positioning of the patient prior to, during, and after treatment. For example, in standard traction therapy, the patient wears a pelvic harness and is positioned supinely (face up) in bed, with the spine slightly flexed and knees bent. Straps or roping which is attached to the harness are then inserted into a pulley mechanism and weights attached at an opposite end, causing a desired pulling/traction effect. Such pulling traction force produces an elongation of the spinal column (distraction) and a reduction in internal intervertebral disc pressure. This creates a vacuum phenomenon inside the disc, which retracts protruded gelatinous material back into its fibrous casing and off of the spinal nerve roots. With the pain gone and the anatomy restored to its natural state, the traction phase of therapy is complete.

An alternate theory for accomplishing the same result is based on extension, rather than flexion of the spine, to achieve reduced intradiscal pressure, while simultaneously anatomically moving nerve roots away from the herniated disc.

While the general principles of flexion and axial traction of the spine are known in the art and have been effected in various strap and/or harness arrangements, either alone or in combination with rotating-pivot type tables as are described above, the inventor has found that both flexion and extension, as well as lateral positioning with traction, can all be beneficial depending upon the patient's particular ailment or condition.

Currently, in the known methods of treating back pain using existing treatment tables the entire lumbar spine, pelvis, and the lower extremity are moved together, which limits the degree of specific angulations of the applied traction force on the spine.

As such, there exists a need for an improved treatment system that is able to move the lower extremity of a patient relatively independently to provide traction to the spine of a patient at varying angles which are not achieved previously. Moreover, it is desirable to combine the positioning and movement of the lower extremity with those of pelvic and lumbar to achieve a wider range of angles of traction.

### SUMMARY OF THE INVENTION

In one aspect, the present invention is directed to a treatment table with a calf/foot assembly, which comprises an upper body support section adapted to support a patient's body, attached to a system support; and a calf/foot assembly pivotally attached to a lower part of the upper body support section, the calf/foot assembly comprising a calf/foot support, a linear movement mechanism adapted to move the calf/foot support linearly in a direction of a longitudinal axis of the upper body support section, and a first rotational mechanism adapted to cause a first transverse movement of the calf/foot support transverse to the longitudinal axis. The calf/foot assembly further comprises a second rotational mechanism adapted to cause a second transverse movement of the calf/foot support transverse to both the longitudinal axis and the first transverse movement. Moreover, the treatment table further comprises a third rotational mechanism adapted to rotate the upper body support section relative to the system support. Additionally, the treatment table also includes a lumbar assembly and a system control adapted to control the linear movement mechanism, the rotational mechanisms and the lumbar assembly.

In a further aspect, the present invention is directed to the method of applying traction to the spine of a patient at varying angles of the treatment table of the present invention. In one embodiment, the method comprises securing the body of a patient on top of the upper body support section, in either supine or prone position, and securing the feet or legs of the patient on the calf/foot support of the calf/foot assembly; and moving the feet and legs of the patient in a first direction left or right relative to a longitudinal axis of the upper body support section to provide traction to the spine of the patient, by actuating the at least one rotational mechanism to cause the first transverse movement of the calf/foot support.

The method further includes moving the calf/foot support linearly along the longitudinal axis to provide a linear traction of the spine, using a linear movement mechanism of the calf/foot assembly adapted to move the calf/foot support linearly.

Furthermore, the method includes remaining the calf/foot support at a position along the first direction, at an angle from the longitudinal axis; and then moving the calf/foot support linearly using the linear movement mechanism of the calf/foot assembly to apply traction to the spine.

Moreover, the method also includes moving the feet and legs of the patient in a second direction transverse to the longitudinal axis and transverse to the first direction to apply further traction to the spine of the patient, by actuating a second rotational mechanism of the treatment table adapted to cause a second transverse movement of the calf/foot support.

Additionally, the method includes remaining the calf/foot support at a position along the first direction, at an angle from the longitudinal axis; remaining the calf/foot support at a position along the second direction, at an angle from the longitudinal axis; and then moving the calf/foot support linearly using the linear movement mechanism of the calf/foot assembly to apply traction to the spine.

With all above described movements, the method can further include adjusting the lumbar assembly in at least one of three dimensions relative to a horizontal plane of the upper body support section to position the lumbar of the patient prior to or during the moving the feet and legs of the patient.

In another embodiment, the present invention provides a method of applying traction to the spine of a patient using the treatment table of the present invention with a pelvic harness. The method includes securing a pelvic harness on the patient; securing the body of the patient on top of the upper body support section, in either supine or prone position, with the feet of the patient rested on the calf/foot support; attaching a belt of the pelvic harness to the calf/foot support; and moving the calf/foot support linearly by actuating the linear movement mechanism of the calf/foot assembly to apply traction to the spine of the patient through the belt.

The method further includes moving the calf/foot support in a first direction transverse to a longitudinal axis of the upper body support section by actuating a first rotational mechanism, and remaining the calf/foot support at a position along the first direction, at an angle from the longitudinal axis; and then moving the calf/foot support linearly using the linear movement mechanism of the calf/foot assembly to apply traction to the spine of the patient through the belt.

Moreover, the method includes moving the calf/foot support in a second direction transverse to the longitudinal axis and transverse to the first direction by actuating a second rotational mechanism of the treatment table, and remaining the calf/foot support at a position along the second direction, at an angle from the longitudinal axis; and then moving the calf/foot support linearly using the linear movement mechanism of the calf/foot assembly to apply traction to the spine of the patient through the belt.

In a further embodiment, the present invention provides a method of providing gravitational traction of the spine of a patient using the treatment table of the present invention. The method includes positioning the upper body support section and the calf/foot assembly in a vertical position relative to a floor; placing the feet of a patient on a foot rest with the body in an upright position, and securing the body of the patient on the upper body support section, in either supine or prone position; and actuating the linear movement mechanism of the calf/foot assembly to descend the foot rest gradually from the feet of a patient, thereby providing gravitational traction of the spine of the patient.

The advantages of the present invention will become apparent from the following description taken in conjunction with the accompanying drawings showing exemplary embodiments of the invention.

### BRIEF DESCRIPTION OF THE DRAWINGS

FIG. 1 is a front perspective view of the inventive multifunction chiropractic treatment table.

FIG. 2 is rear diagonal perspective view thereof.

FIG. 3 is a general side perspective view of the lumbar back support and calf/foot support assemblies.

FIG. 4 is a perspective view showing the vertical positioning of the calf/foot support assembly.

FIG. 5 is a view, similar to that of FIG. 4, however showing the foot support portion elevated relative to the lumbar assembly.

FIG. 6 is a general view of calf and foot support assembly in which the mid section covers are removed, and showing the side-swing motion mechanism of the system.

FIG. 7 is a bottom view of the calf/foot support assembly showing the foot-lock housing thereof attached to the foot side brackets.

FIG. 8 is a side exploded view of the elements of calf/foot support assembly including foot tubing and foot side bracket groups of elements.

FIG. 9 is an exposed view of the interior elements of the calf/foot support assembly.

FIG. 10 is an assembly view of the major components of the calf/foot support assembly.

FIG. 11 are assembly views of the two major subgroups of the foot/calf support assembly.

FIG. 12 is an assembly view, further to FIG. 6, of the elements associated with side-swing motion of the calf/foot support assembly.

FIG. 13 illustrates the method of using the calf/foot assembly in treating a patient in one embodiment of the present invention.

FIG. 14 illustrates the method of using the calf/foot assembly and the pelvic harness in treating a patient at a horizontal position.

FIG. 15 illustrates the method of using the calf/foot assembly and the pelvic harness in treating a patient at a vertical position.

FIG. 16 illustrates the method of using the calf/foot assembly in gravitational traction of a patient's spine.

### DETAIL DESCRIPTION OF THE INVENTION

In one aspect, the present invention provides a treatment table including a calf/foot support assembly for providing traction to the spine of a patient at various angles. In conventional treatment, traction is applied to the patient's spine linearly, or at different angles or from different directions. The force exerted by the traction is referred to as the traction force. Various terms are traditionally used to describe the movements that apply the traction at different angles. Commonly, extension and flexion refer to moving a part of the patient's body backward and forward relative to the spine, respectively. Lateral flexion refers to moving a part of the patient's body left and right relative to the spine.

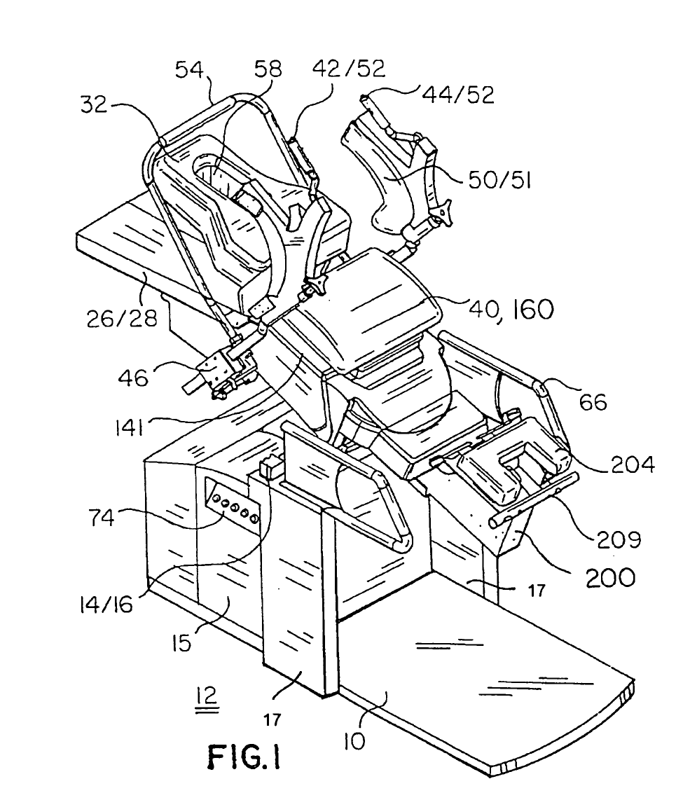

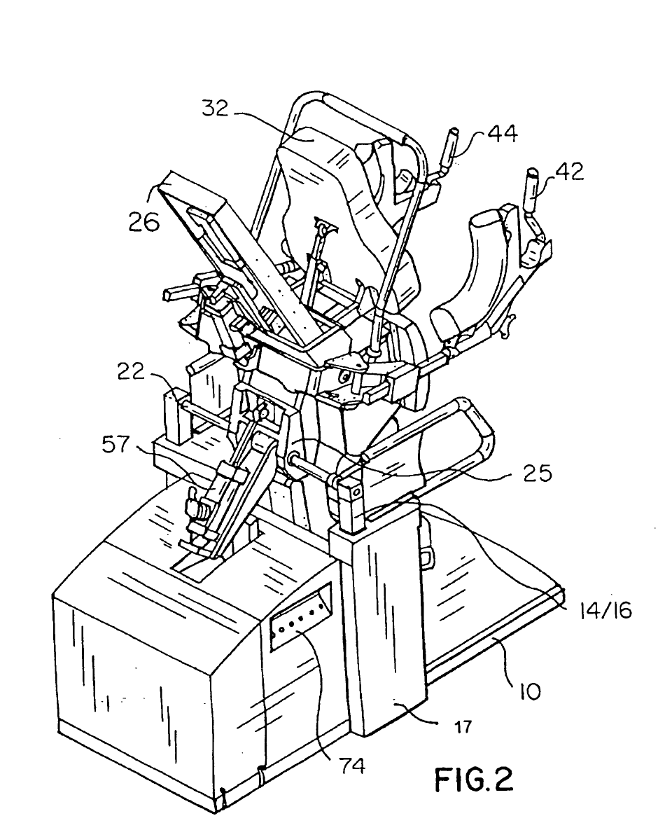

With reference to the views of FIGS. 1 and 2, in one embodiment the treatment table includes a base 10 and a system support 17 in the form of a pair of stands, adapted to rest upon a floor 12 in a typical treatment room of a chiropractor, physical therapist, or other health professionals involved in physical medicine. The treatment table optionally includes a pair of elevation means 14 and 16 on top of system support 17 to enable positioning of patients of various heights on the table. Elevation means 14 and 16 preferably comprise extensible hydraulic pistons, each including upper ends which support a pivot axle 22.

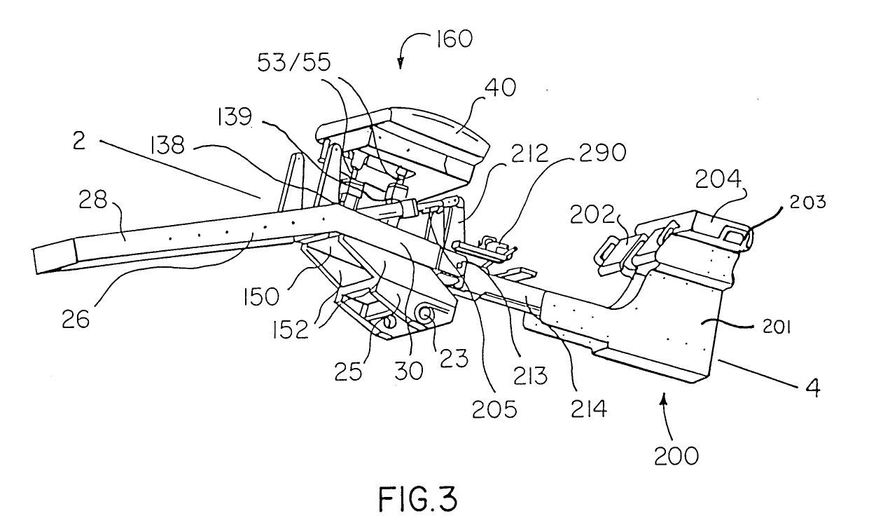

As may be appreciated in the view of FIG. 3, pivot axle 22 is journalled within channel 23 of block support 25 of rigid upper body support platform 26.

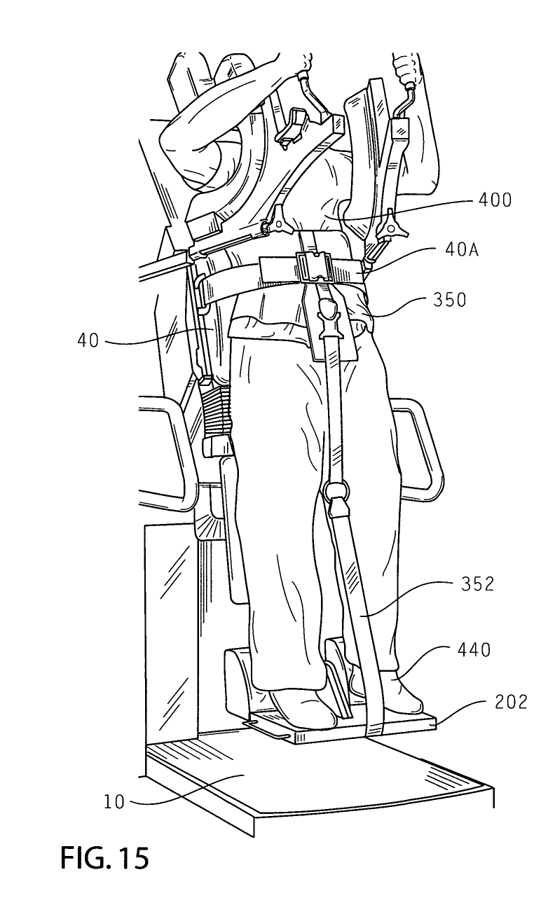

The instant multi-purpose treatment table thereby includes the upper body support platform 26 having an upper part 28 and a lower part 30. The lower part 30 of upper body support platform 26 is secured to the pivot block 25 (see FIG. 3) which is rotatable upon the pivot axle 22 at the approximate mid-point of lower part 30 of platform 26. Pivot axle 22 is disposed on top of and secured to the system support 17. As shown in FIG. 2, the treatment table includes a rotational mechanism 57 adapted to rotate the upper body support platform 26 relative to the base 10 or system support 17. When in use, the upper body support platform 26 can be rotated to a vertical position first (as shown in FIG. 15), the patient can conveniently step on to a foot rest of a calf/foot support assembly as described hereinafter, then the upper body support platform 26 is rotated to a semi-horizontal position, a horizontal position, or slightly beyond the horizontal position with the patient lying above it. The structures and operation mechanism of rotational mechanism 57 have been described in detail in U.S. Pat. No. 6,547,809, which is hereby incorporated by reference in its entirety.

As may be further noted, the upper part 28 defines a plane which is directed at an angle of about thirty degrees relative to a plane defined by the lower part 30 of the upper body support platform 26. Such an angle is necessary in that it allows the patient's upper body to be ergonomically supported by a body support assembly 32 (See FIG. 1), permitting the back to extend convexly and backward relative to base 10 while the patient is in the supine position or permitting the spine to flex in a forward direction while the patient is in the prone position. Body support assembly 32 is mounted upon the upper part 28 of the upper body support platform 26. The body support assembly 32 may or may not be divided into, and may or may not include, moveable sections with hydraulic or pneumatic pistons or other means for elevation and de-elevation of the body support assembly 32. The body support assembly 32 may contain an integral air bladder for additional mobilization and spinal positioning. Herein, the upper body support platform 26 and body support assembly 32 together are referred to the upper body support section, which is adapted to support the upper body of a patient during the treatment.

With reference to FIGS. 1 and 2, the treatment table optionally includes a pair of positionally adjustable arm support means 42 and 44 which are located on top of system support 17, proximally to the sides of a body support assembly 32. As is set forth below, the arm support means include a selectably adjustable rear portion 46 which is secured to the upper part 28 of the upper body support platform 26. The arm support means 42 and 44 include (i) substantially horizontal arm rests 50; (ii) a chest and shoulder support 51 situated posteriorly and angled inwardly in a patient direction from the arm rest; and (iii) a tilted hand grip 52 depending integrally upwardly and inwardly, proximally to the chest and shoulder supports 51.

The treatment table further includes a lumbar and pelvis/buttock support assembly 160, which is displaced from the body support assembly 32. Lumbar cushion 40 is connected to telescoping piston rods 53 and 55 (see FIG. 3) or other means which provide for elevation and de-elevation and rotational positioning in which the top or bottom of the lumbar and pelvis support may be rotated outwardly thereof for direct spinal positioning and localization of applied traction forces to a specific disc and joint level and to provide therapeutic mobilization of the joint and disc. The lumbar cushion 40 may include an internal air cushion in the form of an inflatable air bladder, for added support and tissue mobilization. The same is true of the upper torso support assembly.

As shown in FIG. 1, the treatment table includes a calf/foot support assembly 200, which in short is also referred to as calf/foot assembly. Calf/foot support assembly 200 is pivotally attached to the lower part 30 of the upper body support platform 26, which is more fully described hereinafter.

In FIG. 3 is shown a general view of lumbar assembly 160 and of calf/foot support assembly 200. Therein, FIG. 3 depicts a general position of the lumbar assembly 160 in relation to the calf/foot support assembly 200 and upper body support platform 26, including upper and lower portions of 28 and 30 respectively, associated with the platform 26. As described above, there is an angle between the plane of the upper part 28 and the plane of the lower part 30 of the upper body support platform 26. For the purpose of describing the movements of the calf/foot support assembly 200 relative to the upper body support section, the longitudinal axis 2 of longitudinal axis of said upper body support section is expressed in reference to the lower part 30 of the upper body support platform 26, as the calf/foot support assembly 200 is attached directly to the lower part 30. Depending on the relative position of the calf/foot support assembly, the longitudinal axis 4 of the calf/foot support assembly 200 can be in parallel or in an angle relative to the longitudinal axis 2 of the upper body support platform 26 (see FIGS. 3-5).

Also, shown in FIG. 3 is lumbar cushion 40 and lumbar cushion hydraulic/pneumatic extension rods (or electric screw mechanism or other suitable means) 53/55 which, in combination with hydraulic pistons 138/139, determine the angle of the lumbar cushion 40 relative to lower portion 30 of the upper body support platform 26. Cylinders 138/139 rest upon a rigid support frame plate 150 which itself is supported by a rigid support frame space bar 152 and a block support 25 which includes a main horizontal axial channel 23 through which pivot axle 22 is disposed (see FIGS. 1 and 2).

The treatment table further includes securing means to fasten the patient's body to the treatment table and to offer resistance against the downward force of traction. In one embodiment, belts are provided around the lumbar assembly. Moreover, Velcro™ or other high friction surface with or without belts, pegs, binders, or combinations of these fasten means can be used. Moreover, underarm supports 50 and 51 are also used in some procedures to secure the patient.

The treatment table can change radius of lumbar cushion 40, to effectuate varying degrees of lumbar extension and lumbar support, as well as a general mobilization of the lumbar spine (lower back). Moreover, the lumbar cushion can tilt, and rotate at a variety of angles, allowing a greater range of positions of the patient's lumbar spine, which can be used to increase or decrease the lumbar lordosis and provide direct spinal positioning and localization of applied traction forces to a specific disc or joint level, as well as provide therapeutic mobilization of the joint and disc through repetitive movements of the lumbar cushion. The structures and operation mechanism of the lumbar assembly have been described in detail in U.S. Pat. No. 6,547,809, which is hereby incorporated by reference in its entirety.

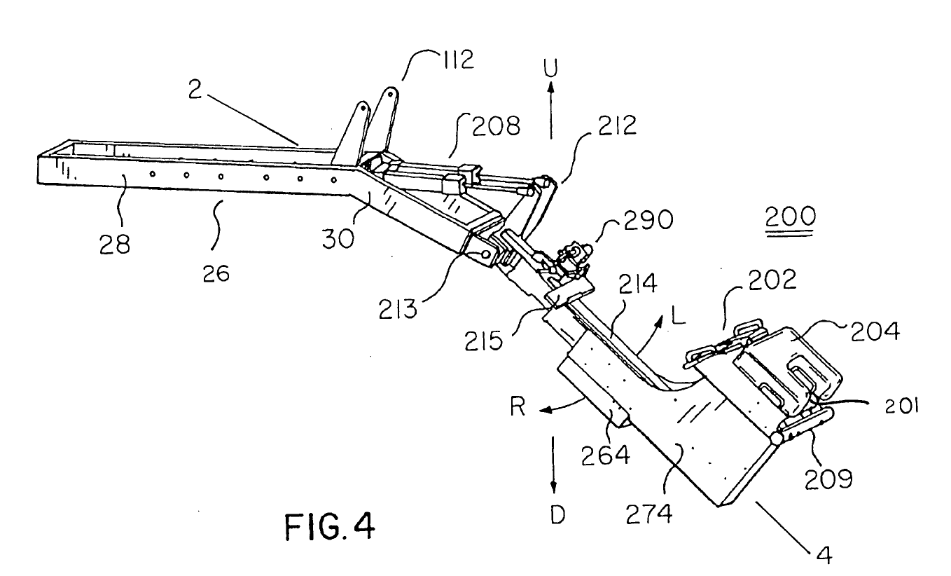

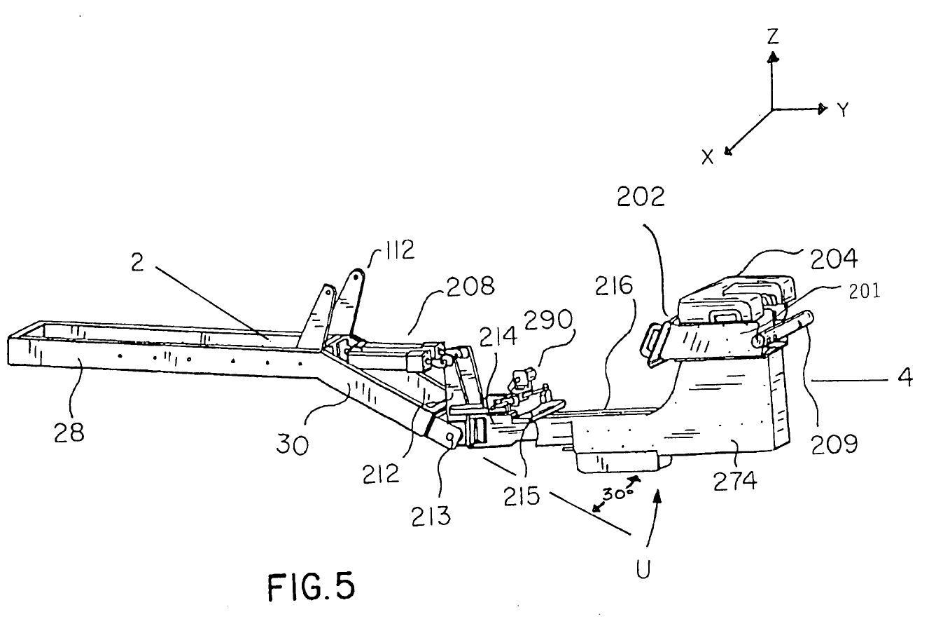

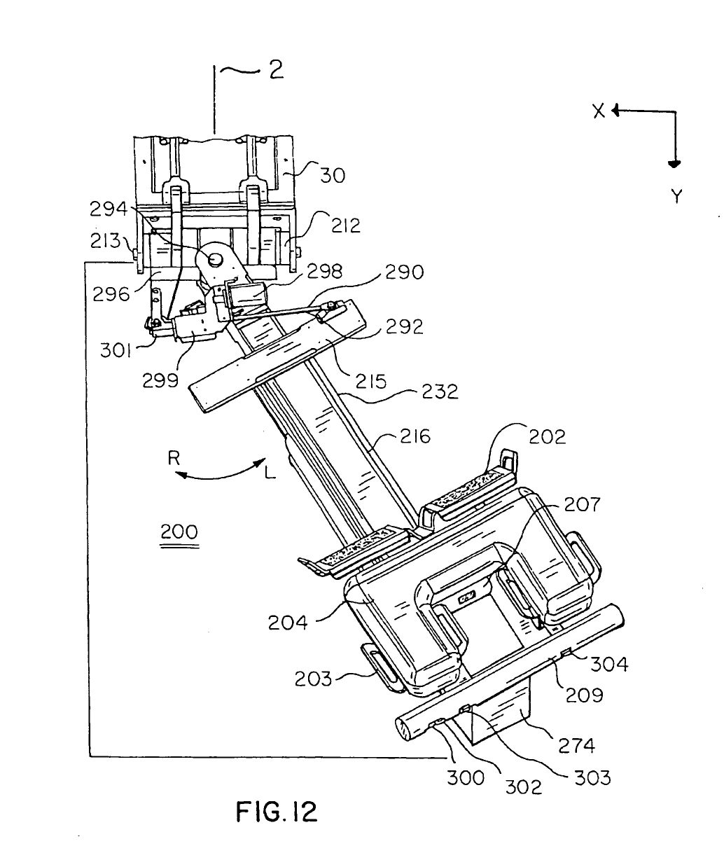

Further shown in FIG. 3 is calf/foot support link casting 212, foot tubing 214, foot rest 202 and calf/foot support 201, all of calf/foot support assembly 200. Also shown therein is side-swing mechanism 290 of calf/foot support assembly 200. Calf/foot support 201 includes an ankle cushion or support 204 and a foot rest 202. The calf/foot support assembly 200 further includes foot fasten means (not shown) attached to the calf/foot support 201 for securing the patient's leg or foot on to calf/foot support 201. In one embodiment, ankle straps are included, which are fastened to the ankle support 204 through ankle strap brackets 203. In another embodiment, a clamping mechanism can be used to secure the patient's leg and ankle, alternatively a boot or sandal may be used which is secured to the calf/foot support 201 or foot platform. In FIG. 4 is a more detailed illustration of the calf/foot support assembly (or calf/foot assembly) and its relationship to upper body support platform 26. More particularly, therein are shown calf/foot support assembly vertical positioning hydraulics 208 and their relationship to the calf/foot support link casting 212 and lumbar support casting 112. Also shown is link casting axle 213 of the calf/foot support link casting 212, the side-swing assembly 290 and its transverse bar 215, foot tubing 214, foot side bracket 274, foot lock housing 264, foot rest 202, ankle cushion 204, and calf/foot support assembly control bar 209. Also shown therein are arrows U, D, R, and L which illustrate the up-down and left-right degrees of freedom of the calf/foot support assembly 200. As can be appreciated, both degrees of freedom are rotational freedom. Reviewing along the longitudinal axis 2 of the upper body support platform 26 (which is along the Y axis in a three-dimensional space, see FIG. 5), the up-down movement is on the Z-Y plane transverse to the longitudinal axis 2; and the left-right movement is on the X-Y plane transverse to longitudinal axis 2 (see FIG. 12).

The calf/foot support assembly 200 is shown in a raised position in FIG. 5. Therein the vertical-positioning hydraulic rods 208 are shown fully retracted, the same corresponding the upper limit of the vertical position (along the Z-axis) of the calf/foot support assembly 200.

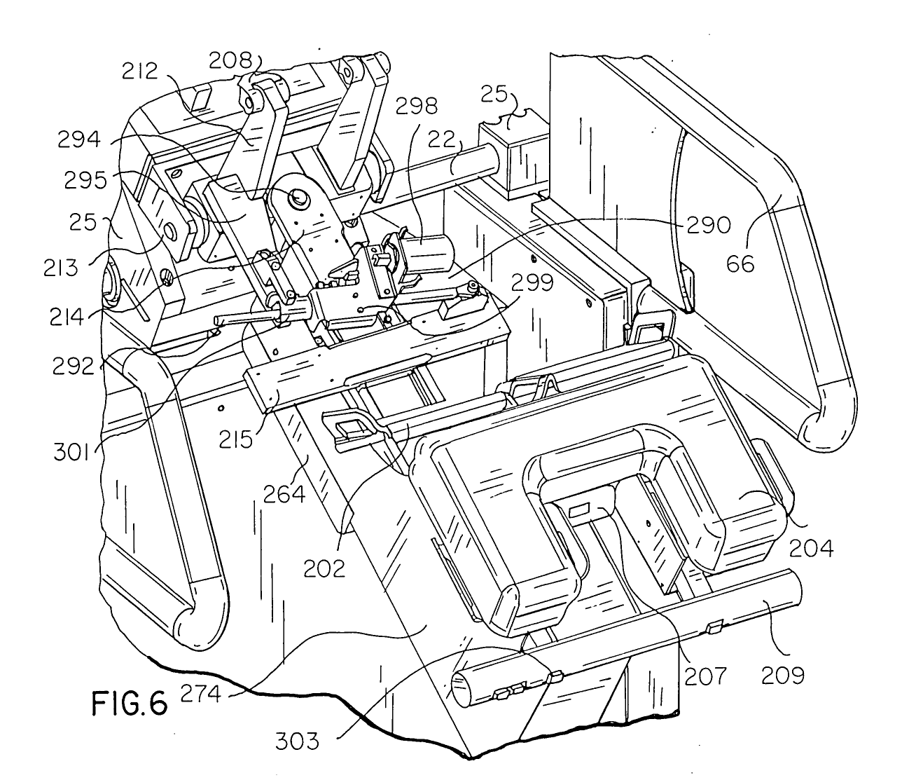

In FIG. 6 is shown a general view of the calf/foot support assembly 200 in which the mid-section covers thereof have been removed. It is noted that the subassemblies of the calf/foot support assembly 200 rest upon foot tubing 214 (see also FIG. 5) that is pivotally attached to the calf/foot support link casting 212, which itself is pivotally attached to the upper body support platform 26 (see axial 213) for vertically positioning, thereby permitting the calf/foot support assembly 200 to be lifted up to 60° above the horizontal plane of lower part 30 of the upper body support platform 26, and moved down as much as 30° therebelow. It is noted that during treatment of a patient the lumbar cushion 40 is typically tilted to lift up the side thereof near the calf/foot assembly 200, therefore the broader range of motion of the calf/foot support assembly 200 in the up direction compensates for the tilting angle of lumbar cushion 40. Alternatively, the calf/foot support 201 can include a hydraulic or other suitable means underneath the ankle cushion 204 to elevate ankle cushion 204 from its support to lift up the patient's feet. This alternative structure can be used for up movement of patient's feet, and can also be used together with the hydraulic rods 208 to achieve the desired up lifting movement described above. In general, in the treatment of a patient the patient's feet can be lifted up to 30° above the patient's upper body, preferably 15° or less, and can be moved down by 30°, preferably 15° or less.

Further shown in FIG. 6 are side handles 66 of the treatment table and, with respect to sided-swing assembly 290, there is shown transverse bar 215, vertical pivot axle 294 which attaches calf/foot support assembly 200 to foot tubing 214, side-swing solenoid 298, and solenoid journal plate 299. Accordingly, upon actuation of the side-swing solenoid 298 by control 303 of the calf/foot support assembly control bar 209, journal plate 299 is rotated to the left upon side swing control bar 292 and, therewith, the calf/foot support assembly 200. Therefore, sided-swing assembly 290 is a rotational mechanism adapted to move the calf/foot support assembly 200 laterally, in other words, causing a transverse movement on the X-Y plane transverse to longitudinal axis 2, which can be more clearly visualized in FIG. 12.

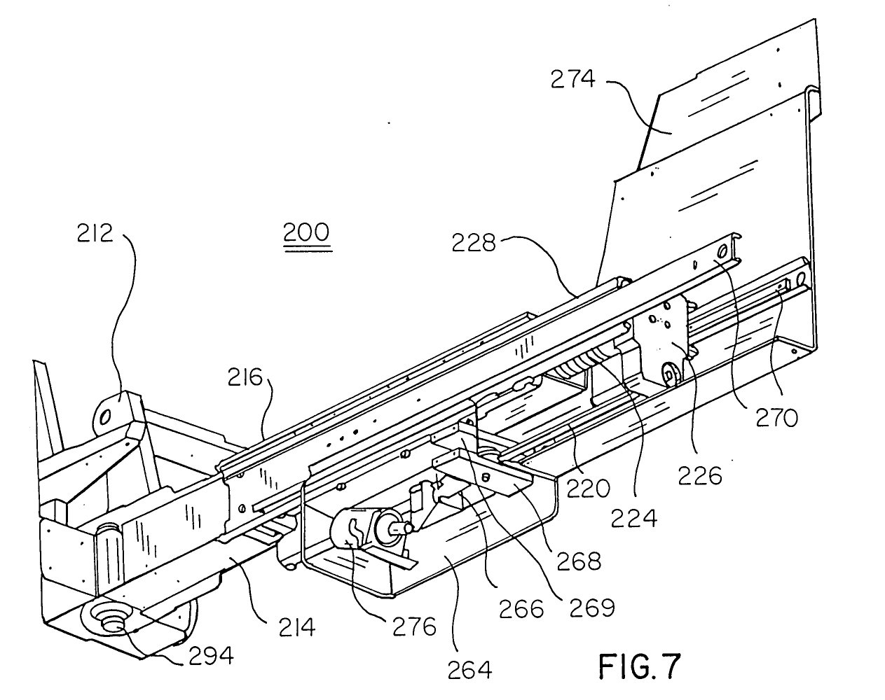

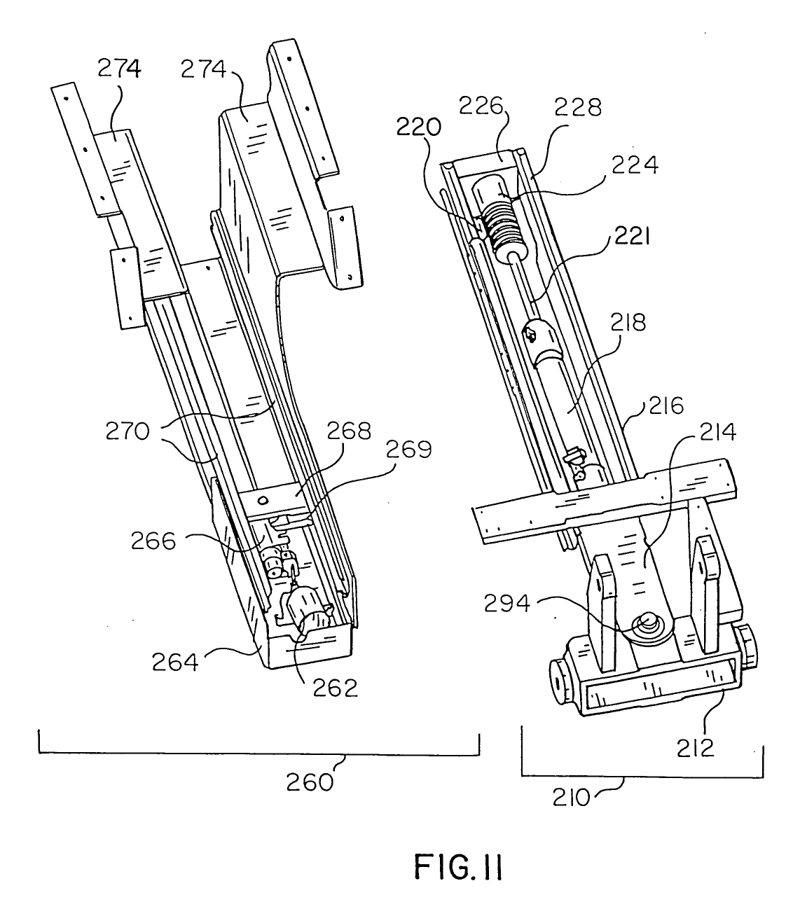

In FIG. 7 is shown the bottom view of the calf/foot support assembly 200 inclusive of foot side brackets 274. In the figure a foot mechanical lock 266 is fixed to foot lock housing 264 by two mechanical lock brackets 268 and 269. A mechanical lock rod 220 is rigidly screwed onto a thrust plate 226 of the calf/foot support assembly 200. When mechanical lock solenoid 276 is activated, foot mechanical lock 266 is released from mechanical lock rod 220, permitting foot-lock housing 264 to slide along the mechanical lock rod 220, thereby adjusting the overall length of the calf/foot support assembly 200, to accommodate different patient heights before the assembly is locked into position. During powered foot traction operation of the system, hydraulic rod 221, spring assembly 224, thrust plate 226 and the entire foot side bracket group 260 (in FIG. 11), move in unison, therefore, all together function as a linear movement mechanism to cause desired linear movement along longitudinal axis 4 of calf/foot support assembly 200.

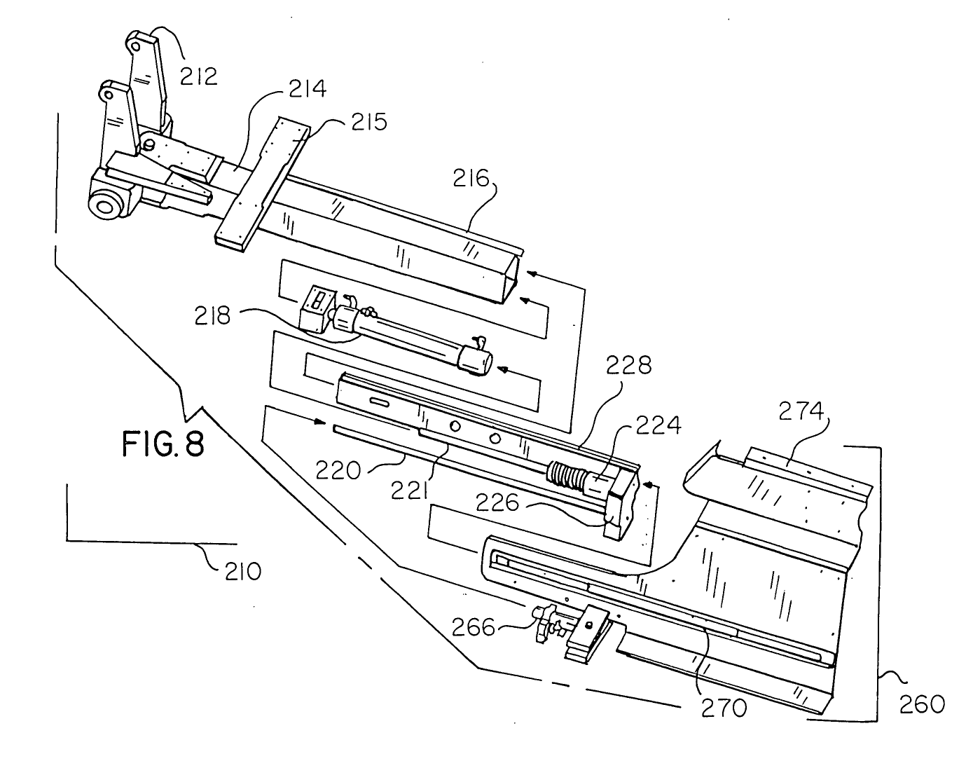

In the exploded view of FIG. 8 may be seen the front half of foot side brackets 274 and middle slides 228. Therein hydraulic cylinder 218, is mounted inside of foot tubing 214, from which hydraulic rod 221 is powered. The other end of the hydraulic rod 221 is rigidly fixed into spring assembly 224 which in turn is mounted upon thrust plate 226. The thrust plate 226 is itself mounted on middle slides 228 for hydraulic/pneumatic linear traction. Middle slides 228 slide upon outer slides 216.

Also attached to thrust plate 226 is the mechanical lock rod 220 which extends in parallel with the rest of foot tubing group 210. Inner slides 270 of foot side bracket group 260 are inserted into the middle slides 228 to enable linear movement. Mechanical lock 266, which is fixed to foot side bracket group 260 through the housing thereof, slides along mechanical lock rod 220 as the foot side bracket group 260 moves linearly relative to the foot tubing group 210. The sliding movement of foot side bracket group 260 is used for patient height adjustment.

After mechanical lock 266 is locked upon mechanical lock rod 220, the foot side bracket group is locked relative to hydraulic rod 221 which can then be used to provide linear traction power to the foot side bracket group 260. Such power traction is linearly guided by the middle slides 228 moving into outer slides 216, as well as by the action of hydraulic cylinder 218 itself, which is a foot traction cylinder.

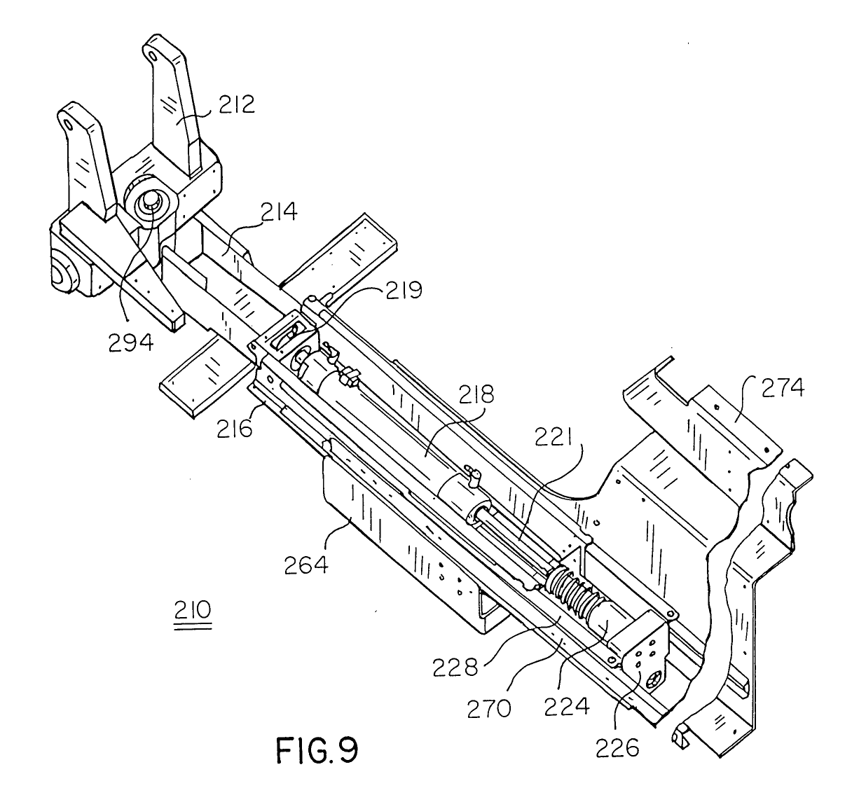

In FIG. 9 is shown an exposed view of calf/foot support assembly 200 including, particularly, the foot tubing group 210 thereof. Foot tubing 214 may be seen, revealing hydraulic cylinder 218 which is located therein. Further shown is one of foot side brackets 274. It may be further seen that hydraulic rod 221 is connected to shock absorbing spring assembly 224 that is attached to thrust plate 226 which, in turn, is mounted upon middle slides 228 upon each side thereof. Inner slides 270 are fixed to the inside of foot side brackets 274. Outer slides 216 are externally attached to the sides of foot tubing 214. Middle slides 228 and inner slides 270 are indirectly locked by a mechanical lock in the foot lock housing 264 to insure transfer of hydraulic power to the foot side brackets. Also shown in FIG. 9 is load cell 219 which monitors over-pressure conditions at a proximal output of hydraulic cylinder 218, and vertical pivot axle 294 upon which foot tubing 214 rotates.

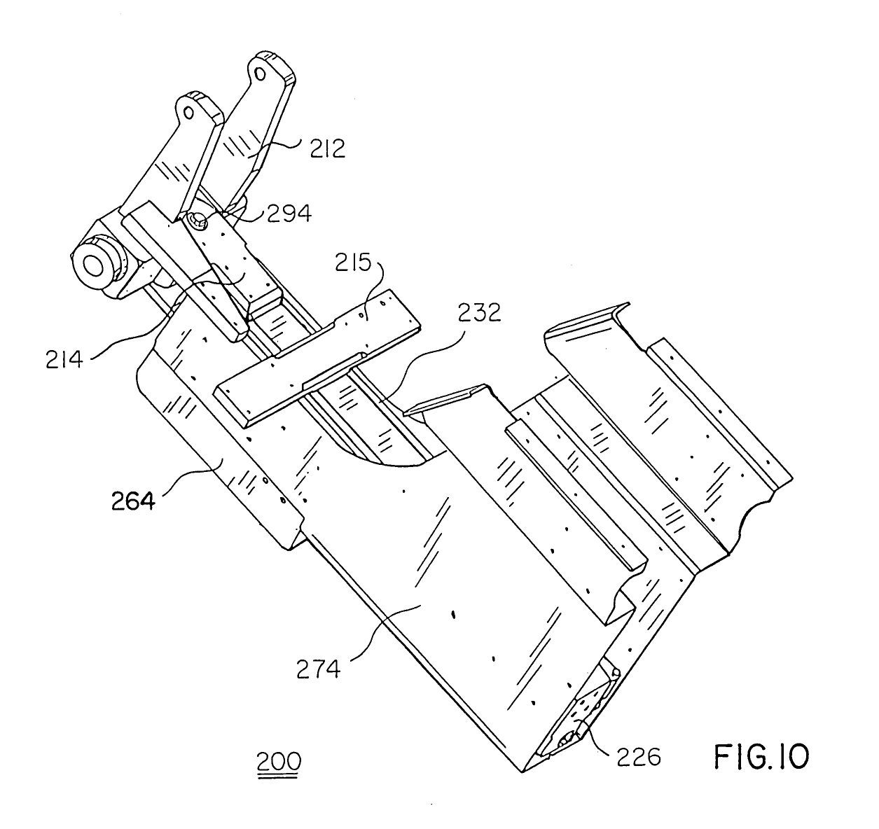

In FIG. 10 is shown major components of calf/foot support assembly 200 including foot/calf support link casting 212, vertical pivot axle 294, foot tubing 214, transverse bar 215, ball-bearing slide assembly 232, foot lock housing 264, foot side brackets 274, and thrust plate 226. Therein, foot tubing 214 houses a foot traction hydraulic assembly (not shown) and pivots horizontally upon vertical pivot axle 294 of foot/calf support link casting 212. Ankle cushion, foot support assembly control bar (both not shown), and ankle harnesses mount on the foot side brackets and are slidably mounted upon foot tubing 214 by ball bearing slide assembly 232. As noted in FIG. 7, a mechanical locking assembly is located inside of foot lock housing 264 and operates to unlock the main components of the foot assembly when patient height adjustment is required.

In FIG. 11 is shown foot tubing group 210 and foot side bracket group 260. Therein, the foot side bracket group 260 is pivotally attached to the foot support link casting 212 by vertical axial 294, and houses the foot traction hydraulic assembly inclusive of hydraulic cylinder 218, hydraulic rod 221, and spring assembly 224.

Foot side bracket group 260 is slidably attached to the foot tubing group 210 by means of the inner slides 270, middle slides 228, and outer slides 216. Individual adjustments to accommodate patient's heights are made by allowing mechanical lock 266 to slide upon mechanical lock rod 220, thus locking foot mechanical lock to the mechanical lock rod after an adjustment of relative position of foot side bracket group 260 to the hydraulic rod 220 of the foot tubing group 210. Alternatively, patient's height may be accommodated by increasing overall length of traction cylinder 218 and other suitable means to provide adjustment of patient's height.

It is noted that the linear movement of calf/foot support assembly 200 along its longitudinal axis 4 has been described above with the embodiment of a hydraulic mechanism; however, it should be understood that various other mechanisms enabling actuating a linear movement can also be used for the purpose of the present invention. One example is a screw driven mechanism powered by a motor.

FIG. 12, which is related to FIG. 6, is an illustration of the side-swing motion and side-swing assembly 290 of the calf/foot support assembly 200. Therein calf/foot support assembly is pivotally attached to foot/calf support link casting 212 at vertical pivot axle 294 to effect sideway (left and right) movements of the calf and leg. The side-swing assembly 290 (described more fully above in FIG. 6) links to foot tubing 214 through vertical pivot axle 294 which connects solenoid journal plate 299 of the side-swing assembly 290 to transverse bar 215 which rests upon ball bearing slide assembly 232 (See also FIG. 10). Thereby, actuation of side-swing solenoid 298 by side motion control switch 303 will govern of the left to right motion of the calf/foot support assembly 200. Once locked by side-swing mechanical lock 301, the relative side-swing angle of the calf/foot support assembly 200 relative to lower part 30 is maintained.

As also shown in FIG. 12 are the controls associated with calf/foot support assembly control bar 209, namely, power traction Y axis control switches 300 and 302 as well as patient height, Y axis adjustment switch 304. Also shown in FIG. 12 is LCD reader 207, a foot rest 202, ankle cushion 204, and ankle strap brackets 203.

It is to be understood that FIGS. 1 and 2 further illustrates a system control 74 for use by the doctor which includes function buttons for moving the treatment table or a subassembly thereof, such as those shown in Table 1.

TABLE 1

| | | |
|---|---|---|
| 1. | TBL LFT = | Table Lift. |
| 2. | TBL LWR = | Table Lower. |
| 3. | ROT BACK = | Rotate Table Back. |
| 4. | ROT FWD = | Rotate Table Forward. |
| 5. | ARM UP = | Translational Arm Height Up. |
| 6. | ARM DWN = | Translational Arm Height Down. |
| 7. | OPEN | |
| 8. | OPEN | |
| 9. | ARM R. UP = | Arm Rotate Up. |
| 10. | ARM R. DOWN = | Arm Rotate Down. |
| 11. | LUM IN = | Lumbar In |
| 12. | LUM OUT = | Lumbar Out |
| 13. | OPEN | |
| 14. | OPEN | |
| 15. | RBK TL = | Rotate Table Back with Table Lift. |
| 16. | RFW TLW = | Rotate Table Forward with Table |
| | | and Lower Table. |
| 17. | SAFETY ON | A safety on and off button is |
| | AND OFF = | included which stops pneumatic/hydraulic |
| | | piston and ceases all table movement. |

As a safety measure, controls may also be incorporated into overhead gripping means 54 or into handgrips 42 (see FIGS. 1 and 2), with optional patient control of other functions.

More preferably, the system control comprises a processor, which can include preprogrammed automated single or multiple actions, and preprogrammed automated patient treatment protocols. Furthermore, the system control can comprise a display screen, such as a LCD monitor, or touch screen application as a user interface. The control system further includes a pressure sensor to record and provide feedback to the amount of traction force applied and displayed on a LCD screen or a computer monitor, which offers the practitioner immediate feedback on traction force applied. The system control can also be voice activated.

In a further aspect, the present invention provides methods of treating a patient using the treatment of the present invention described above.

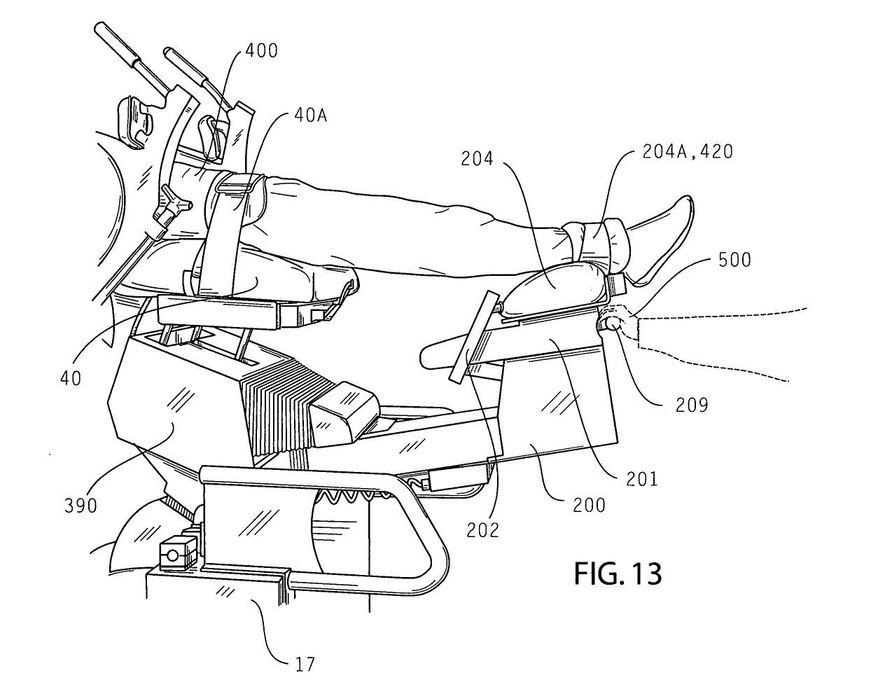

In one embodiment, the method involves one or more treatment process that applies traction to a patient's spine at varying angles. Before the treatment, the body of the patient is secured to the upper body support section by belts or other suitable fasten means, more specifically on top the upper body support 32 and lumbar cushion 40, in either supine or prone position, the patient's ankles or feet are secured on to the calf/foot support 201 of calf/foot assembly 200. It is noted that for convenient access, typically the rotational mechanism 57 is actuated to rotate the upper body support section to a vertical position relative to the floor 12, and the patient steps on to the foot rest 202 of calf/foot assembly 200. It is noted that the foot rest 202 can be rotated over the ankle cushion for approximately 180°, or slightly beyond. At the loading position, foot rest 202 is at the lowest extreme of the calf/foot assembly 200 below ankle cushion 204 (as shown in FIG. 15), and once the patient is rotated to a horizontal position using rotational mechanism 57, the foot rest 202 is moved around the ankle cushion 204 and stays in the opposite side of the calf/foot assembly control bar 209. In one exemplary embodiment as shown in FIG. 13, the patient's body 400 is fastened to lumbar cushion 40 by a belt 40A that is attached to lumbar assembly 160, and the patient's ankle 420 are fastened to ankle cushion 204 by an ankle strap 204A attached to ankle strap brackets 203. As shown in FIG. 13, at the horizontal position the upper body support section and the calf/foot assembly are above the system support 17. A cover 390 covers the mechanical components between the lower part 30 of the upper body support platform 26 and the calf/foot assembly 200.

The treatment of applying traction to a patient's spine at varying angles can be performed at several levels. In one embodiment, at the horizontal position described above, the sided-swing assembly 290, also referred to as the first rotational mechanism, is actuated to move the calf/foot support 201, hence, the feet and legs of the patient, left or right relative to the longitudinal axis 2 of the upper body support section, as can be appreciated in FIG. 12. For the purpose distinguishing from other transverse or lateral movement, the left or right direction is referred as the first direction and the left or right movement is also referred to as the first transverse movement. As can be appreciated, such a movement provides a traction force useful in lateral flexion of the patient's spine at a range of angles from 0 to about 45° from longitudinal axis 2, preferably less than 30°. As shown in FIG. 13, the left or right movement can be controlled manually by the doctor's hand 500 using the function buttons provided on the calf/foot assembly control bar 209. Alternatively, it can also be controlled by the system control.

Moreover, the method further involves moving the calf/foot support 201 linearly to provide linear traction of the spine, using a linear movement mechanism of the calf/foot assembly. Therefore, the patient can be treated using different functionalities of the calf/foot assembly, which provide different types and combinations of delivery of traction forces, such as linear vs. lateral.

In a further embodiment, the method combines these different functionalities to apply traction at varying angles not achievable by only individual functions. More specifically, the treatment process involves remaining the calf/foot support 201 at a position along the first direction (left or right), at an angle from the longitudinal axis 2, and then, moving the calf/foot support 201 linearly using the linear movement mechanism of the calf/foot assembly 200 to provides traction of the spine. As can be appreciated, because of the preposition of calf/foot support 201 at an angle from the longitudinal axis 2, the traction force generated by the linear movement of calf/foot support 201 is applied to the spine at an angle. This angle can be determined by the doctor according to the patient's condition and the desired treatment protocol. The effect of applying linear traction at a predetermined angle is substantially different from the prior art method which only enables a linear traction along longitudinal axis 2, which is also the longitudinal axis of the patient's body. Alternatively, a linear traction can be provided first using the linear movement mechanism of the calf/foot assembly 200, during application of the linear traction of calf/foot support 201 can be moved left or right relative to the longitudinal axis 2 to provide a dynamic movement in a range of angles.

In another embodiment, the method involves moving the feet and legs of the patient in a second direction which is transverse to the longitudinal axis 2 and transverse to the first direction by actuating the vertical positioning hydraulics 208, which is herein also referred to as the second rotational mechanism, to cause a second transverse movement of the calf/foot support 201. The second transverse movement is illustrated in FIGS. 4 and 5, where it is referred to as up or down movement, as can be appreciated when the upper body support section and the calf/foot assembly are in the horizontal position relative to the floor.

In a further embodiment, the method further combines the two transverse movements with the linear movement described above. More specifically, the treatment process involves remaining or locking the calf/foot support 201 at a position along the first direction (left or right), at an angle from the longitudinal axis 2, and also remaining or locking the calf/foot support 201 at a position along the second direction (up or down), at an angle from longitudinal axis 2, then moving the calf/foot support 201 linearly using the linear movement mechanism of the calf/foot assembly to provide traction at further angles which are not achievable using only one transverse movement described. As can be appreciated, the traction force created in this manner can be selectively focused to a specific disc and joint level, which can address the need to accommodate a specific pain-relieving anatomical treatment position, which includes lateral and medial sciatic antalgia positioning during application of linear traction. Moreover, as can be appreciated that the two transverse movements can also be provided without using the linear traction of the calf/foot assembly 200.

Alternatively, a linear traction can be provided first using the linear movement mechanism of the calf/foot assembly 200, during the application of the linear traction the calf/foot support 201 can also be moved left or right, and up and down relative to the longitudinal axis 2 to provide a dynamic movement in a range of angles.

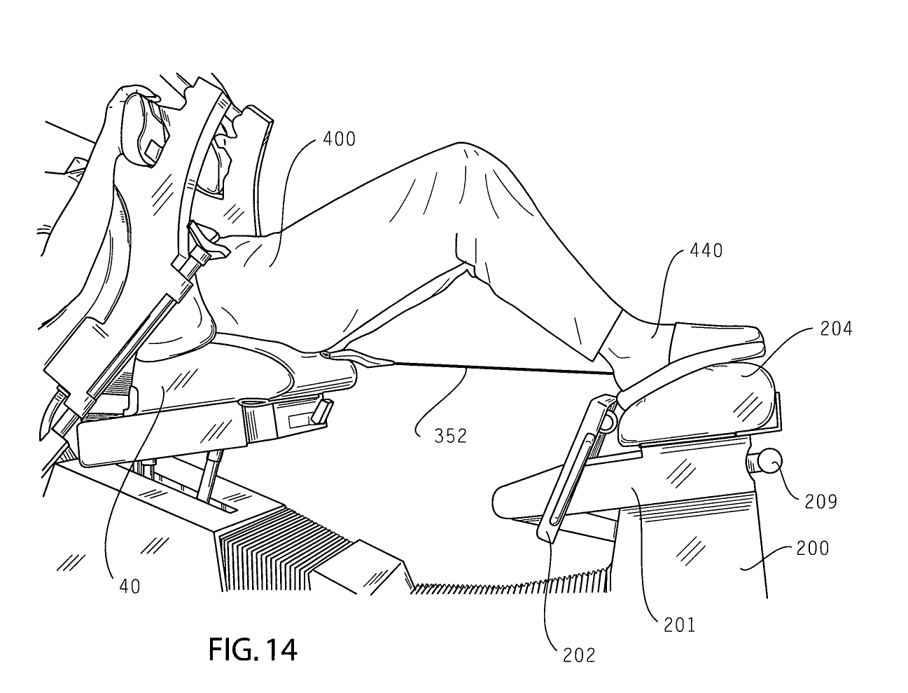

In another embodiment, an alternative approach is used to apply traction to the patient's spine with the use of a pelvic harness 350, as illustrated in FIGS. 14 and 15. In this treatment process, the patient wears a pelvic harness (can be seen in FIG. 15), prior to being placed on the treatment table. After securing the patient to the upper body support section, the patient is rotated to the horizontal position as described above. It is noted that the patient can be secured to the lumbar cushion 40 by fasten means, or being supported by the underarm supports of the treatment table or by attaching to the overhead bar. In this process, the patient's feet 440 are rested on the ankle cushion 204 or on foot rest 202 and the patient's legs are relaxed, which can be either stretched or bent as shown in FIG. 14. The belt 352 of the pelvic harness 350 is attached to the calf/foot support 201 of the calf/foot assembly 200. Then, the linear movement mechanism of the calf/foot assembly 200 is actuated to move the calf/foot support 201 linearly away from the upper body support section, which applies traction to the spine through the pelvic harness. As can be appreciated, different from the embodiment described above, in this approach the patient's legs are relaxed and the traction force is applied through the pelvic harness 350, not through the legs. It should be understood that in this embodiment the first and second transverse movements described above can also be used for applying traction at varying angles.

This alternative approach can also be used when the upper body support section and the patient are in the vertical or semivertical position. FIG. 15 shows a patient under such a treatment in a vertical position. As shown, the patient wears pelvic harness 350 and the belt 352 is attached to foot rest 202. In this position, when the linear movement mechanism of the calf/foot assembly 200 is actuated, the calf/foot support 201 descends away from the upper body support section, which applies traction force to the spine through the pelvic harness. As can be appreciated, in the vertical position ankle straps or foot restraints may be used alternatively without wearing of the pelvic harness. Moreover, the first and second transverse movements described above can also be used for applying traction at varying angles.

It is noted that the traction force created by the first transverse movement, the linear movement, or the linear movement at an angle, can be static (i.e. constant application of a force during a period of time) or intermittent (application of greater force for a period of time followed by a lesser force for a period of time).

Additionally, all above described process can be further combined with the use the lumbar assembly of the treatment table. More specifically, lumbar assembly 160 is actuated to adjust the position or orientation of lumbar cushion in one or more dimensions relative to a horizontal plane of the lower part 30 of the upper body support platform 26 to position the patient's lumbar prior to or during the transverse movements and/or linear movement. The adjustment increases or decreases the degree of lumbar spine flexion and extension, increasing or decreasing the lumbar lordosis, and effectuating posterior pelvic or anterior rotation, which further controls the location of the traction force to be applied to the patient, and further enhances the therapeutic effect of the treatment.

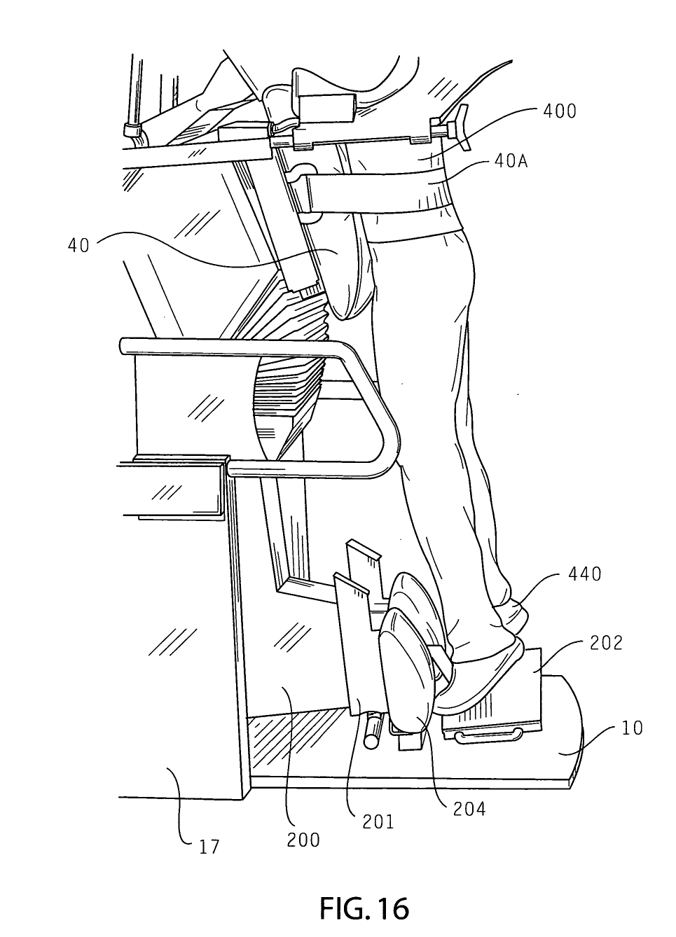

In another embodiment, the method of the present invention provides gravitational traction of the spine of a patient using the present treatment table. In this process, the upper body support section and the calf/foot assembly 200 are in a vertical position relative to the floor, adjusted using the rotational mechanism 57. The patient steps on to the foot rest 202 of calf/foot support 201 at its loading position described above, and patient's body, in either supine or prone position, is secured to the upper body support section using underarm supports and combination of thoracic restraint and lumbar strap. Then, the upper body support section together with the calf/foot assembly 200 is rotated to an angle, about 5° to 40° away form the vertical position. Subsequently, the linear movement mechanism of the calf/foot assembly 200 is actuated to descend the foot rest 202 gradually until sufficient weight or unloading of the spine is accomplished, this may include having the patient remain in partial contact with foot rest 202, or for maximum gravitational traction, foot rest 202 disconnects completely from the feet of a patient. In the latter situation, the patient's body is no longer supported from underneath, the body weight provides a maximum gravitational traction of the spine of the patient. FIG. 16 shows a patient in the process of this treatment right before the foot rest 202 completely disconnects from the patient's feet 440. Moreover, with the feet disconnected from the foot rest, alterations in rotation of upper body support platform 26 or the degree of rotation from the vertical to the horizontal also increases or decreases gravity effects, as more or less of body weight of patient is supported by patient support frame and cushions. It is further noted that prior to the foot rest 202 completely disconnects from the patient's feet, the first and second transverse movements described above can also be used for applying traction at varying angles.

Furthermore, the second rotational mechanism, or vertical positioning hydraulics 2081 is actuated to move the calf/foot support 201 in a direction front or back to the longitudinal axis 2. It is noted that this front or back movement is the same to the second transverse movement of up or down when the upper body support section and the calf/foot assembly are in the horizontal position. When the calf/foot support 201 is moved front or back, ankle cushion 204 is in contact with the patient's legs. As can be appreciated, this transverse movement changes the angle of the traction force from that experienced by the patient in the absence of the transverse movement or positioning.

Additionally, in this embodiment the lumbar cushion 40 can also adjusted in one or more dimensions to position the lumbar of the patient prior to, during or after descending the foot rest. The positioning of lumbar cushion further modifies the gravitational traction to the patient's spine.

It is noted that the above described linear and transverse movements can be actuated using the control buttons on the calf/foot support assembly control bar 209, or can be actuated using the programmed functions displayed on the system control screen. Typically, information required for lumbar treatment, such as treatment time, force increase time, force decrease time, hold time, rest time, maximum force, and mode of the treatment such as static or intermittent are entered for generating the treatment protocols.

As can be appreciated, the methods described above using the present treatment table produce the forces and positions required to cause decompression of the intervertebral discs. In other words, it achieves a rapid lengthening of the muscles and longitudinal ligaments of the spine, increasing the separation of the intervertebral disc and articular joint spaces. This results in both mobilization of the spine and rapid development through the “disc unloading” of a negative internal disc pressure responsible for causing the vacuum phenomenon for retracting protruding disc material back within the borders of a healthy disc. The treatment table of the present invention can be used to treat many conditions, including, but not limited to, back pain, protruding disc, herniated disc, degenerative disc disease, posterior facet syndrome and sciatica.

The method of the present invention using the calf/foot assembly to provide traction at various angles has several advantages over the prior art methods.

Currently, in the known methods of treating back pain using existing treatment tables the entire lumbar spine, pelvis, and the lower extremity are moved together on the same plane, which limits the degree of specific angulations of the applied traction force on the spine and restricts the ability to preposition of specific spinal segments (disc and vertebrae). Using the method of the present invention, the movement of the lower extremity is relatively independent from the lumbar spine and pelvis. As described above, prior to and during the application of traction, selected positioning of the calf/leg assembly is made. The selected positioning of lower extremity provides a certain spinal positions which are not achieved by the existing methods. Moreover, it has been found that the combination of selected positioning of the calf/leg assembly and selected positioning of the lumbar assembly provides a greater range of spinal positions in extension, flexion, circumduction, lateral flexion, and axial positioning in the prone or supine position, in other words, a wider range of angles or direction of traction is accomplished. For example, rotating the lumbar support and moving the calf/leg assembly downward direction provides a greater degree and ability to place the spine in greater angle for applied traction.

Furthermore, independent operation of the calf/leg assembly and the lumbar assembly isolate the spatial orientation of the pelvis and lumbar spine from the direction of traction. As can be appreciated, the positioning of lumbar spine to induce traction and the delivery of traction through the linear movement of the calf/leg assembly are relatively separated. The lumbar spine is positioned and stabilized with strapping, and the calf/leg assembly applies traction force independently, providing improved treatment stability, as well as versatility.

Additionally, in the prior art method the lower extremity supports can be only moved up or down manually, which limits the degree and precision of application of the traction force, and is difficult to reproduce and control gradual change of the force. Moreover, it increases effort and practitioner's fatigue. Using the treatment table of the present invention, the powered transverse movements (left/right and up/down) of the calf/foot assembly controlled by hydraulic, pneumatic, and electric system provides precise control of desired traction force to the patient. Furthermore, treatment time, change of the force, rate of change can all be precisely controlled by the powered system.

As described above, the calf/foot assembly control bar is located next to the ankle cushion. Various functions of operating the calf/foot assembly are built into the control bar for doctors to use during the treatment. For example, lifting the control bar will initiate powered positioning of calf/foot support 201 in an upward or downward direction, and the X-AXIS Release control button controls movement of the calf/foot support 201 in the left/right direction. Alternatively, these functions can also be provided by the system control.

While there has been shown and described the preferred embodiment of the instant invention it is to be appreciated that the invention may be embodied otherwise than is herein specifically shown and described and that, within said embodiment, certain changes may be made in the form and arrangement of the parts without departing from the underlying ideas or principles of this invention.

I claim:

1. A treatment table with a calf/foot assembly comprising:
(a) an upper body support section adapted to support a patient's body, attached to a system support; and
(b) a calf/foot assembly attached to a lower part of said upper body support section, said calf/foot assembly comprising a calf/foot support, a linear movement mechanism adapted to move said calf/foot support linearly in a direction of a longitudinal axis of said upper body support section, and a first rotational mechanism adapted to cause a first transverse movement of said calf/foot support transverse to said longitudinal axis.

2. The treatment table of claim 1, wherein said calf/foot assembly further comprises a second rotational mechanism adapted to cause a second transverse movement of said calf/foot support transverse to both said longitudinal axis and said first transverse movement.

3. The treatment table of claim 2, wherein said second rotational mechanism pivotally connects said calf/foot assembly to said upper body support section.

4. The treatment table of claim 3, wherein said second transverse movement is in a range from 45° above a plane of said lower part of said upper body support section to 30° below said plane.

5. The treatment table of claim 4, wherein said calf/foot assembly further comprises a calf/foot assembly control bar.

6. The treatment table of claim 5 further comprising a lumbar assembly disposed on said lower part of said upper body support section.

7. The treatment table of claim 6, wherein said lumbar assembly comprises moving mechanisms enabling adjustments of said lumbar assembly in three dimensions.

8. The treatment table of claim 7 further comprising a third rotational mechanism adapted to rotate said upper body support section relative to said system support.

9. The treatment table of claim 8 further comprising a system control adapted to control said linear movement mechanism, said first, said second and said third rotational mechanisms and said moving mechanisms of said lumbar assembly.

10. The treatment table of claim 9, wherein said system control comprises a processor adapted to maintain a plurality of treatment protocols.

11. A method of applying traction to the spine of a patient at varying angles, comprising:
(a) providing a treatment table comprising an upper body support section adapted to support a patient's body and attached to a system support; a calf/foot assembly attached to said upper body support section, said calf/foot assembly comprising a calf/foot support; and at least one rotational mechanism adapted to cause a first transverse movement of said calf/foot support;
(b) securing the body of the patient on top of said upper body support section, in either supine or prone position, and securing the feet or legs of the patient on said calf/foot support of said calf/foot assembly; and
(c) moving the feet and legs of the patient in a first direction left or right relative to a longitudinal axis of said upper body support section to provide traction to the spine of the patient, by actuating said at least one rotational mechanism to cause said first transverse movement of said calf/foot support.

12. The method of claim 11 further comprising:
moving said calf/foot support linearly along said longitudinal axis to provide a linear traction of the spine, using a linear movement mechanism of said calf/foot assembly adapted to move said calf/foot support linearly.

13. The method of claim 11 further comprising:
adjusting a lumbar assembly disposed at a lower part of said upper body support section in at least one of three dimensions relative to a horizontal plane of said upper body support section to position the lumbar of the patient prior to or during said moving the feet and legs of the patient.

14. The method of claim 11 further comprising:
remaining said calf/foot support at a position along said first direction, at an angle from said longitudinal axis; and
then moving said calf/foot support linearly using said linear movement mechanism of said calf/foot assembly to apply further traction to the spine.

15. The method of claim 11 further comprising:
moving the feet and legs of the patient in a second direction transverse to said longitudinal axis and transverse to said first direction to apply further traction to the spine of the patient, by actuating a second rotational mechanism of said treatment table adapted to cause a second transverse movement of said calf/foot support.

16. The method of claim 15 further comprising:
moving said calf/foot support linearly along said longitudinal axis to provide a linear traction of the spine, using a linear movement mechanism of said calf/foot assembly adapted to move said calf/foot support linearly.

17. The method of claim 16 further comprising:
adjusting a lumbar assembly disposed at a lower part of said upper body support section in at least one of three dimensions relative to a horizontal plane of said upper body support section to position the lumbar of the patient prior to or during said moving the feet and legs of the patient.

18. The method of claim 16 further comprising:
remaining said calf/foot support at a position along said first direction, at an angle from said longitudinal axis;
remaining said calf/foot support at a position along said second direction, at an angle from said longitudinal axis; and
then moving said calf/foot support linearly using said linear movement mechanism of said calf/foot assembly to apply further traction to the spine.

19. A method of applying traction to the spine of a patient comprising:
(a) providing a treatment table comprising an upper body support section adapted to support a patient's body and attached to a system support; a calf/foot assembly attached to said upper body support section, said calf/foot assembly comprising a calf/foot support and a linear movement mechanism adapted to move said calf/foot support linearly;
(b) securing a pelvic harness on the patient;
(c) securing the body of the patient on top of said upper body support section, in either supine or prone position, with the feet of the patient rested on said calf/foot support;
(d) attaching a belt of said pelvic harness to said calf/foot support; and
(e) moving said calf/foot support linearly by actuating said linear movement mechanism of said calf/foot assembly to apply traction to the spine of the patient through said belt.

20. The method of claim 19 further comprising:
moving said calf/foot support in a first direction transverse to a longitudinal axis of said upper body support section by actuating a first rotational mechanism, and remaining said calf/foot support at a position along said first direction, at an angle from said longitudinal axis; and
then moving said calf/foot support linearly using said linear movement mechanism of said calf/foot assembly to apply traction to the spine of the patient through said belt.

21. The method of claim 20 further comprising:
moving said calf/foot support in a second direction transverse to said longitudinal axis and transverse to said first direction by actuating a second rotational mechanism of said treatment table, and remaining said calf/foot support at a position along said second direction, at an angle from said longitudinal axis; and
then moving said calf/foot support linearly using said linear movement mechanism of said calf/foot assembly to apply traction to the spine of the patient through said belt.

22. The method of claim 21 further comprising:
adjusting a lumbar assembly disposed at a lower part of said upper body support section in at least one of three dimensions relative to a horizontal plane of said upper body support section to position the lumbar of the patient prior to or during said moving said calf/foot support.

23. A method of providing gravitational traction of the spine of a patient comprising:
(a) providing a treatment table comprising an upper body support section adapted to support a patient's body and attached to a system support; a calf/foot assembly attached to said upper body support section, said calf/foot assembly comprising a calf/foot support and a linear movement mechanism; a rotational mechanism adapted to rotate said upper body support section and said calf/foot assembly;
(b) positioning said upper body support section and said calf/foot assembly in a vertical position relative to a floor using said rotational mechanism;
(c) placing the feet of a patient on a foot rest of said calf/foot support with the body in an upright position, and securing the body of the patient on said upper body support section, in either supine or prone position, by securing means; and
(d) actuating said linear movement mechanism of said calf/foot assembly to descend said foot rest gradually from the feet of a patient, thereby providing gravitational traction of the spine of the patient.

24. The method of claim 23 further comprising:
adjusting a lumbar assembly disposed at a lower part of said upper body support section in at least one of three dimensions relative to a horizontal plane of said upper body support section to position the lumbar of the patient prior to, during or after said descend said foot rest.

25. The method of claim 24 further comprising:
moving the legs of the patient front or back relative to a longitudinal axis of said upper body support section to provide further traction at angles, by actuating a further rotational mechanism to move said calf/foot support transversely to said longitudinal axis.
